(19) United States
(12) Patent Application Publication
(10) Pub. No.: US 2006/0166885 A1
(43) Pub. Date: Jul. 27, 2006

(54) Treatment and prevention of decubitus

(75) Inventor: Dirk Willem Van Bekkum, Rotterdam (NL)

Correspondence Address: TRASK BRITT, P.O. BOX 2550, SALT LAKE CITY, UT, 84110, US

(73) Assignee: Crucell Holland B.V., Leiden (NL)

(21) Appl. No.: 11/386,995

(22) Filed: Mar. 22, 2006

Related U.S. Application Data
(63) Continuation of application No. PCT/EP04/52545, filed on Oct. 14, 2004.

(30) Foreign Application Priority Data
Oct. 17, 2003 (WO) PCT/EP03/50733

(51) Int. Cl. A61K 38/18

(52) U.S. Cl. 514/12

(57) ABSTRACT

Provided is a method for treating a subject suffering from, or at risk of suffering from, decubitus, the method comprising a step of administering erythropoietin (EPO), or a functional part, derivative or analogue thereof to the subject. In certain embodiments, the EPO has been recombinantly produced in host cells that further express the E1A protein of an adenovirus.

### CROSS-REFERENCE TO RELATED APPLICATION

This application is a continuation of PCT International Patent Application No. PCT/EP2004/052545, filed on Oct. 14, 2004, designating the United States of America, and published, in English, as PCT International Publication No. WO 2005/037304 A1 on Apr. 28, 2005, which claims priority to PCT International Patent Application No. PCT/EP03/50733, filed on Oct. 17, 2003, the contents of the entirety of each of which are hereby incorporated herein by this reference.

### FIELD OF THE INVENTION

The present invention relates generally to the fields of biotechnology and medicine. More, in particular, the invention relates to therapies that aim to treat and/or prevent decubitus.

### BACKGROUND OF THE INVENTION

Decubitus (pressure ulcers, pressure sores, bed sores) develops in the skin and subcutaneous tissues that are subjected to prolonged pressure as occurs in areas that carry weight of the body in bedridden patients. The clinical symptoms of pressure sores can be divided into four stages (Oxford Handbook of Clinical Specialities, 5th Edition, 1999): Stage I: non-blanching erythema over intact skin; Stage II: partial thickness skin loss, for example, shallow crater; Stage III: full thickness skin loss, extending into fat; and Stage IV: destruction of muscle, bone, or tendons. Preventive measures consist of the avoidance of pressure points anywhere on the body surface by use of air and water cushions, mattresses, regular change of position of the body and the limbs, and stimulation of the circulation in the areas at risk. Treatment of decubitus is often difficult and the lesions can be very painful. The prevention and treatment of decubitus poses a large burden on nursing. Seven percent of inpatients have pressure sores. Up to 85% of paraplegic patients have pressure sores. The emphasis is on prevention, as treatment of existing ulcers is time consuming and cumbersome. Therefore, a need exists for further therapies for the prevention and/or treatment of decubitus. The present invention aims at providing such therapies.

### SUMMARY OF THE INVENTION

Described is a method for treating a subject suffering from, or at risk of suffering from, decubitus ulcers, the method comprising a step of administering erythropoietin, or a functional part, derivative or analogue thereof to the subject. The invention provides for use of erythropoietin or a functional part, derivative or analogue thereof for the treatment and/or prevention of decubitus ulcers. The invention provides a use of erythropoietin or a functional part, derivative or analogue thereof for the preparation of a medicament for treatment and/or prevention of decubitus ulcers. In certain embodiments, the erythropoietin, functional fragment, derivative or analogue thereof, has been recombinantly produced in host cells that further express the E1A protein of an adenovirus, preferably PER.C6® cells.

In another aspect, the invention provides a pharmaceutical preparation for the treatment or prevention of decubitus ulcers, characterized in that the preparation comprises erythropoietin, or a functional part, derivative or analogue thereof. In certain embodiments hereof, the pharmaceutical preparation is suitable for topical administration.

### BRIEF DESCRIPTION OF THE FIGURES

FIG. 1: Surface of ulcers developing after eight hours of pressure in MF-1 mice (Example 5A). Values are means; bars represent standard deviations; t(d) means time (days); squares represent buffer; and triangles represent EPO treated. 3.3 μg EPO was administered intraperitoneally one hour before the application of and immediately after the release of pressure.

FIG. 2: Surface of ulcers developing after ten hours of pressure in Balb/c mice (Example 5B). Values are means; bars represent standard deviations; t(d) means time (days); squares represent buffer; and triangles represent EPO treated. 4.5 μg EPO was administered intraperitoneally one hour before the application of pressure.

FIG. 3: Surface of ulcers developing after six hours of pressure in MF-1 mice (Example 5C). Values are means; bars represent standard deviations; t(d) means time (days); squares represent buffer; and triangles represent EPO treated. 4.5 μg EPO was administered intravenously ten minutes before the application of pressure.

### DETAILED DESCRIPTION OF THE INVENTION

For the present invention, the definition of decubitus is meant to include the development, or risk of development, of skin ulcers due to prolonged pressure on an area of the skin, and the terms “pressure sores,” “pressure ulcers” and “bed sores” are meant to be included into the definition of decubitus. The definition includes all stages of the conditions indicated by these terms, such as the four stages described in the background of the invention hereinabove, starting with non-blanching erythema over intact skin.

In the present invention, EPO is used for treating subjects suffering from, or at risk of suffering from, decubitus ulcers. In certain embodiments of the invention, the treatment of a subject with erythropoietin is aimed at avoiding effects of EPO believed to be undesired when treating or preventing conditions of decubitus ulcers. To that effect, EPO variants, fragments, derivatives or analogs are used that have decreased erythropoietic effect as such or due to pharmacokinetic properties. In one preferred embodiment, the EPO contains Lewis-X structures on its N-linked glycans, preferably on average at least about 1.5 Lewis-X structures per EPO molecule. In accordance with one preferred method, the EPO is a low sialylated version of EPO, preferably having on average less than ten, more preferably less than eight, still more preferably less than six sialic acid moieties per EPO molecule. In accordance with another preferred embodiment, such EPO variant is obtainable by recombinantly producing EPO in a cell that is characterized by low sialylation of recombinant proteins produced therein. Examples of such cells are adenovirus E1-expressing cells. In one preferred embodiment thereof, the cells are cells derived from PER.C6 cells, expressing E1 and EPO, or a fragment, derivative or analog thereof. In certain embodiments, the treated subjects are not anemic.

The present invention for the first time discloses the use of EPO for the treatment of decubitus, irrespective of whether the hematocrit value (red blood cell count) of the patient is lower than normal or not. This provides decubitus per se as a novel indication for the use of EPO. The present invention, therefore, provides for the use of EPO for treatment of patients with decubitus, wherein the patients do not necessarily have another indication besides decubitus, which would otherwise have warranted the treatment of such a patient with EPO based on the presently available knowledge. A “non-anemic patient” as used herein, is a patient that has a hemoglobin value that is considered as being within the normal range, which value would not otherwise lead a physician to prescribe EPO to this patient, i.e., absent knowledge of the current invention. Up till now, application of EPO is mostly restricted to the prevention or correction of anemia in specific patient populations, including the (pre)dialysis phase of chronic renal insufficiency, cytostatic therapy, premature infants and as preparation for autologous blood transfusion or surgical procedures with anticipated major blood loss. The general aim in such cases is to increase hemoglobin levels (Hb) by increasing the number of red blood cells (hematocrit) to a specific range by adapting standard dosage regimes to individual needs. Depending on the patient population, the optimal Hb level ranges from a lower limit of 6.5 to 7.5 mmol/L to an upper limit of 8.0 to 8.7 mmol/L. Hence, in certain embodiments of the present invention, the subjects treated with EPO for decubitus are not patients in the (pre)dialysis phase of chronic renal insufficiency, undergoing cytostatic therapy, premature infants, or patients undergoing autologous blood transfusion or surgical procedures with anticipated major blood loss.

EP Patent Application No. 1072609 relates to cytoprotective agents comprising prosaposin-related peptides, and describes that peptides referred to as 18-MP already at concentrations in the picomolar to femtomolar range, promote expression of BCl-X_{L} protein, and suppress apoptosis-like neuron death. Based on these observations, a wide range of clinical applications for 18-MP is envisaged, including treatment of peripheral tissue diseases accompanied by apoptosis or apoptosis-like cell death. Bedsore is one of the conditions mentioned. Based on the finding that EPO enhances expression of BCl-X_{L} protein at similarly low doses as well, it is suggested that EPO has the same effect and efficacy as 18-MP. No proof of the action of 18-MP for treatment of decubitus is presented in EP 1072609. Furthermore, it should be noted that not all apoptosis involves the BCl-X_{L} pathway. In any case, the molecular reasons for the clinical symptoms of decubitus are complex and presently not completely understood, and the involvement of apoptosis in the clinical symptoms of decubitus is unclear, if existent. On the contrary, decubitus is characterized at certain stages by necrosis (e.g., J. A. Witkowski and L. C. Parish (1982), J. Am. Acad. Dermatol. 6:1014-1021). Necrosis is clearly distinct from apoptosis (e.g., R. Paus et al. (1993), Exp. Dermatol. 2:3-11). In fact, apoptosis has been reported to appear concurrently with reepithelialization of the wound and may signal the end of the inflammatory phase of healing at that site in the wound (Brown et al. (1997), Surgery 121:372-380) and, therefore, induction of apoptosis might even be beneficial rather than causal to skin wounds. A link between apoptosis and decubitus, therefore, appears fully unclear. In addition, a causal relationship between any agent inducing Bcl-X_{L} and treatment and/or prevention of decubitus appears to include at least one extra level of uncertainty. The present invention for the first time provides the use of EPO for treatment and/or prevention of decubitus.

A “subject,” according to the present invention may be an animal and in preferred aspects, is a human subject.

Erythropoietin (EPO) is a glycoprotein hormone, which in humans has a molecular weight of 34 to 38 kD. The glycosyl residues comprise about 40% of the molecule. The role of EPO that has been studied and put to practice most is in the production of red blood cells. Recently, other uses have been envisaged for EPO, such as the protection, restoration and enhancement of EPO-responsive cells (PCT International Patent Publication WO 02/053580, the contents of which are incorporated by this reference). It is the merit of the present invention to describe the novel use of EPO for the treatment or prevention of decubitus ulcers.

Many forms of EPO, as well as functional fragments, derivatives and analogues thereof have been described, and it will be clear that all these are included within the scope of the present invention for the prevention and/or treatment of decubitus ulcers.

“EPO,” according to the invention, is EPO as may be isolated from any suitable source. Preferably, human EPO is recombinantly produced and isolated from a suitable recombinant host cell and/or from the culture medium. In the case of recombinant production, the host may suitably be chosen from any cell capable of recombinantly producing protein, such as bacterial host cells (e.g., E. coli, B. subtilis), yeast (e.g., S. cerevisiae, K. lactis), fungi (e.g., A. niger, Pichia), mammalian cells (e.g., CHO, BHK) including human cells. According to one aspect of the invention, EPO is recombinantly produced in a human cell line, in particular, an immortalized human embryonic retina cell line expressing E1A of an adenovirus, such as a PER.C6 cell line. It is also possible to administer EPO in a gene therapy setting according to the invention, for instance, by treating a patient with a vector comprising a nucleic acid sequence capable of expressing EPO when delivered to a target cell.

Derivatives of EPO refer to modifications of the source EPO, which may be urinary EPO, or EPO recombinantly producible from a cDNA or gene sequence, wherein the expression product has one or more modifications relative to the source EPO, which modifications may be in the primary structure, by substitution of one or more amino acid residues (such as in Novel Erythropoiesis Stimulating Protein (NESP)), deletion, addition or relocation of one or more amino acid residues, or alterations in the post- or peri-translational modification of the protein backbone, such as hydroxylations, phosphorylations or glycosylations of amino acid residues, sulfur bridges, and the like. Derivatives also encompass naturally or non-naturally occurring EPO variants coupled to non-EPO-related proteinaceous moieties or even to non-proteinaceous moieties. EPO and derivatives useful for the present invention include both native erythropoietins as well as erythropoietins that have been altered by at least one modification as compared to native erythropoietin, and preferably as compared to native human erythropoietin. The at least one modification may be a modification of at least one amino acid of the erythropoietin molecule, or a modification of at least one carbohydrate of the erythropoietin molecule.

Erythropoietin molecules useful for the purposes herein may have a plurality of modifications compared to the native molecule, such as multiple modifications of the amino acid portion of the molecule, multiple modifications of the carbohydrate portion of the molecule, or at least one modification of the amino acid portion of the molecule and at least one modification of the carbohydrate portion of the molecule. Derivatives of EPO are encompassed by the instant invention, as long as they interact with the EPO receptor and cause a reduction or prevention of decubitus. This can be tested by methods known to the person skilled in the art, such as those according to the examples provided herein.

Functional analogues of EPO refer to molecules not necessarily derived from naturally or non-naturally occurring EPO, that are capable of mimicking the interaction of EPO with its receptor, whereby decubitus is reduced and/or prevented. Such functional analogues may comprise peptidomimetics and/or non-peptidic molecules mimicking the idiotope interacting with the EPO-R. It will be understood by those of skill in the art, that the functional analogue according to the invention need not necessarily interact with the same idiotope, or in the same way, as long as it mimics the interaction of EPO with its receptor. Functional analogues may suitably be screened and selected from (synthetic) peptide libraries, phage or ribosome polypeptide display libraries, or small molecule libraries. Those of skill in the art are capable of screening for or designing functional analogues and test their functionality in assays disclosed herein.

The forms of EPO useful in the practice of the present invention encompass naturally occurring, synthetic and recombinant forms of erythropoietin, including, but not limited to, urinary EPO, brain EPO, renal EPO, serum EPO, etc. By way of non-limiting example, forms of EPO useful for the practice of the present invention include EPO muteins, such as those with altered amino acids at the carboxy terminus described in U.S. Pat. Nos. 5,457,089 and 4,835,260; agonists described in U.S. Pat. No. 5,767,078; EPO isoforms with various numbers of sialic residues per molecule, such as described in U.S. Pat. No. 5,856,292; polypeptides described in U.S. Pat. No. 4,703,008; peptides which bind to the EPO receptor as described in U.S. Pat. Nos. 5,773,569 and 5,830,851; small-molecule mimetics which activate the EPO receptor, as described in U.S. Pat. No. 5,835,382; and EPO analogues as described in WO 95/05465, WO 97/18318 and WO 98/18926. All these patent citations are incorporated herein by this reference, especially to the extent that such disclosures refer to the various alternate forms or processes for preparing such forms of EPO of the present invention. EPO can be obtained commercially (for instance, under the trademarks of PROCRIT, available from Ortho Biotech; EPOGEN, available from Amgen, Inc.; and EPREX, available from Jansen-Cilag).

Glycosylation of the EPO molecule can have impact on its functionality. Any glycosylation form of EPO is encompassed in the present invention. EPO that is now commercially available and used for increasing the red blood cell counts (hematocrit) of patients in need thereof can be used according to the invention. In certain embodiments, however, the EPO used for the present invention has less erythropoietic activity. In one embodiment, the EPO of the invention has at least no sialic acid moieties. In another embodiment, the modified EPO is asialoerythropoietin. In another embodiment, the modified EPO has 1, 2, 3, 4, 5, 6, 7, 8, 9, 10, 11, 12, or 13 sialic acid moieties. In another embodiment, the modified EPO has at least no N-linked or no O-linked carbohydrates. In another embodiment, the modified EPO has at least a reduced carbohydrate content by virtue of treatment of EPO with its native carbohydrates with at least one glycosidase, or by virtue of having a mutation in at least one of the amino acids to which an N-linked or O-linked carbohydrate is bound in natural EPO. In another embodiment, the modified EPO has less N-linked tetra-antennary carbohydrate chains than natural EPO.

It will be immediately clear that an EPO molecule according to the invention can conveniently be produced in recombinant host cells, wherein genetic information encoding EPO is present in expressible format. Molecular biology provides convenient methods well known to the person skilled in the art for generating the genetic information that encodes any kind of EPO, fragments, derivatives or analogues thereof. The expression of recombinant nucleic acid molecules in host cells is also well known in the art. This provides the advantage of an easy to manipulate, renewable source of EPO with the same characteristics, in contrast to purification of EPO from natural sources, which may be more burdensome. It will, therefore, be immediately clear that wherever a functional fragment or derivative is described by deleting and/or mutating and/or adding amino acids from/to EPO, this can be conveniently brought about by deletions and/or mutations and/or additions to the EPO coding sequence that is used for recombinant expression. Hence, in certain embodiments, the EPO, or functional fragment, derivative or analogue thereof is recombinantly expressed in a host cell. In certain embodiments thereof, the host cell is a cell that expresses the E1A protein of an adenovirus. Preferably, the cell is derived from a human retina cell. Most preferably, the cell is derived from a PER.C6 cell. Recombinant production of EPO in PER.C6 cells has been described in WO 00/63403, which is incorporated by reference herein to the extent that it describes methods for the production of EPO in PER.C6 cells. EPO that is produced in host cells that express the E1A protein of an adenovirus has a specific glycosylation pattern, described in WO 03/038100, herewith incorporated by reference in its entirety. This EPO has less erythropoietic activity than commercially available EPREX. A low erythropoietic activity may be an advantage for the treatment of decubitus patients with low mobility, as an increase in hematocrit for such patients could be seen as an undesired side-effect of EPO. In certain preferred aspects of the invention, therefore, used EPO molecules comprise on average at least 0.5 Lewis-X structures (which may include sialyl-Lewis-X structures) per N-linked glycan (i.e., at least 1.5 Lewis-X structures per EPO molecule). Preferably, in these aspects, the EPO molecules on average comprise less than ten sialic acid moieties, more preferably, less than eight, still more preferably, less than six sialic acid moieties per EPO molecule.

EPO, according to the present invention, can be of any source, but preferably is human EPO when human subjects are to be treated. The use of human EPO may prevent an immunogenic response against the administered protein. Of course, human EPO encompasses EPO protein that has been obtained from human tissue or fluid, such as blood or urine, as well as EPO that has been recombinantly produced in host cells, wherein the nucleic acid sequence encoding the EPO is a sequence encoding human EPO. Preferably, the host cells are human cells.

In certain embodiments of the invention, EPO is administered in a gene therapy setting, e.g., nucleic acid comprising the coding sequence of EPO is administered, for example, in a viral vector, such as an adenovirus vector which includes the EPO coding sequence in expressible format. Administration of the nucleic acid encoding EPO, for example, transdermally, intradermally, or systemically, and the like, to a subject suffering from or at risk of suffering from decubitus, will result in EPO being expressed and having its therapeutic and/or prophylactic effect in the treated subject. The person skilled in the art knows how to prepare gene delivery vehicles, such as recombinant adenovirus, comprising nucleic acid sequences encoding EPO, a derivative, fragment or analogue thereof, and administer these to a subject, for example, by local injection. It will, therefore, be clear to the skilled person that a gene therapy setting wherein nucleic acid comprising the coding sequence of EPO in expressible format is administered to a subject is included within the meaning of “administering EPO” according to the present invention.

According to the present invention, EPO can be administered to patients at risk of developing decubitus ulcers, prior to the manifestation of symptoms, to preclude the development of ulcers. Alternatively, or additionally, according to the invention, EPO may be administered to patients soon after the manifestation of clinical symptoms, such as redness and painfulness of the affected body parts. Further, according to the invention, EPO may be administered to patients that already suffer from decubitus ulcers, to improve their condition. In preferred embodiments, the EPO is administered in the early stages of the process, such as stage I characterized by the non-blanching erythema over intact skin. According to another preferred embodiment, a patient at high risk for developing pressure sores is treated prophylactically, e.g., before clinical symptoms are observed, with EPO on a regular basis.

According to the invention, EPO may be administered systemically, for instance, intravenously, or by local injection, for instance, subcutaneously, or topically, or in any other form known to the person skilled in the art.

Treatment regimes for erythropoietic purposes are well established. In general, EPO dosages are given in IU (international units), referring to the activity of EPO in erythropoiesis. Such IU correlate to the protein content of EPO but are operationally defined and, hence, the correlation may vary between different batches. As a rule of thumb, one IU corresponds to 8 to 10 ng epoetin alfa. It will be clear to the person skilled in the art that although the IU are usually given for commercial EPO preparations, the concentration of EPO molecules in such preparations can easily be defined according to standard procedures. This will allow determination of the relative specific activity, for example, in IU/g (see, for example, EP 0428267). In certain embodiments, the administered dose of EPO or EPO equivalent, according to the present invention, is in the range of 0.1 to 1,000,000 IU per kg body weight, preferably in the range of 1 to 10,000 IU per kg body weight, when administered intravenously or subcutaneously. The dose, for instance, depends on the route of administration. This may, of course, also depend on the form of EPO used, for example, on the erythropoietic potential of the used EPO. Normal doses of EPO that are administered to adult renal failure patients are in the range of 4000 to 7500 IU per week (80 to 100 kg body weight). These amounts are normally divided into three separate doses per week for the commercially available epoetin alpha or Eprex (EPO produced on CHO cells). For the present invention, such doses are suitable. In other embodiments, higher doses may be given daily or even more frequently. The maximum tolerable dose may have to be determined in order to prevent hematocrit values and hemoglobin concentrations to rise too sharply. Persons of ordinary skill know how to monitor hematocrit values and hemoglobin concentrations in patients to prevent undesired side effects, such as extreme high blood pressure that may occur in later stages of the treatment. For improved results, it may be required to administer EPO for a longer period. For this purpose, doses may range from 10 to 100,000 IU per administration, preferably 1000 to 2500 IU per administration (for an adult of 80 to 100 kg). Also, in this case, monitoring may be necessary to prevent unwanted side effects. For less erythropoietic EPO, such as EPO that has been recombinantly produced in PER.C6 cells, higher doses may be administered, for example, 5000 IU/kg.

EPO, according to the invention, may be in the form of a pharmaceutically acceptable composition as known to the person skilled in the art. The compositions may comprise inter alia stabilizing molecules, such as albumin or polyethylene glycol, or salts. Preferably, the salts used are salts that retain the desired biological activity of EPO and do not impart any undesired toxicological effects. Examples of such salts include, but are not limited to, acid addition salts and base addition salts. Acid addition salts include, but are not limited to, those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include, but are not limited to, those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. If necessary, EPO may be coated in or on a material to protect it from the action of acids or other natural or non-natural conditions that may inactivate the EPO.

Furthermore, the present invention pertains to pharmaceutical compositions comprising at least EPO, a functional fragment, derivative or analogue according to the invention. The pharmaceutical composition of the invention further comprises at least one pharmaceutically acceptable excipient. A pharmaceutical composition according to the invention can further comprise at least one other therapeutic and/or prophylactic agent. These may include an anti-inflammatory agent, such as hydrocortisone, prednisone and the like. They may also include analgesic agents such as salicylic acid, acetaminophen, ibuprofen, flurbiprofen, morphine and the like. Such agents are all well known to the skilled person. Preferably, the further therapeutic and/or prophylactic agents are agents capable of preventing and/or treating decubitus ulcers. Typically, pharmaceutical compositions are sterile and stable under the conditions of manufacture and storage. The EPO can be in powder form for reconstitution in the appropriate pharmaceutically acceptable excipient before or at the time of delivery. In the case of sterile powders for the preparation of sterile injectable solutions, the preferred methods of preparation are vacuum drying and freeze drying (lyophilization) that yield a powder of the active ingredient plus any additional desired ingredient from a previously sterile-filtered solution thereof. Alternatively, the EPO can be in a solution and the appropriate pharmaceutically acceptable excipient can be added and/or mixed before or at the time of delivery to provide a unit dosage injectable form. Preferably, the pharmaceutically acceptable excipient used in the present invention is suitable to high drug concentration, can maintain proper fluidity and, if necessary, can delay absorption.

The choice of the optimal route of administration of the pharmaceutical compositions will be influenced by several factors including the physico-chemical properties of the active molecules within the compositions, the urgency of the clinical situation and the relationship of the plasma concentrations of the active molecules to the desired therapeutic effect. For instance, if necessary, the EPO of the invention can be prepared with carriers that will protect it against rapid release, such as a controlled release formulation, including implants, transdermal patches, and microencapsulated delivery systems. Biodegradable, biocompatible polymers can be used, inter alia, such as ethylene vinyl acetate, polyanhydrides, polyglycolic acid, collagen, polyorthoesters, and polylactic acid. Furthermore, it may be necessary to coat the EPO with, or co-administer the EPO with, a material or compound that prevents the inactivation of the EPO. For example, the EPO may be administered to a subject in an appropriate carrier, for example, liposomes, or a diluent.

The routes of administration of the EPO, according to the present invention, include, but are not limited to, bolus, oral, buccal, epidermal, inhalation, intra-arterial, intradermal, intramuscular, intraperitoneal, intrasternal, intravenous, subcutaneous, topical, etc. Preferred administration routes, according to the invention, include intravenous injection or infusion or local injection at the site of the lesion. In another preferred embodiment, EPO is administered topically to the skin.

The pharmaceutical compositions of the present invention can also be formulated for parenteral administration. Formulations for parenteral administration can be, inter alia, in the form of aqueous or non-aqueous isotonic sterile non-toxic injection or infusion solutions or suspensions. The solutions or suspensions may comprise agents that are non-toxic to recipients at the dosages and concentrations employed, such as 1,3-butanediol, Ringer's solution, Hank's solution, isotonic sodium chloride solution, oils such as synthetic mono- or diglycerides, or fatty acids such as oleic acid, local anesthetic agents, preservatives, buffers, viscosity or solubility-increasing agents, water-soluble antioxidants such as ascorbic acid, cysteine hydrochloride, sodium bisulfate, sodium metabisulfite, sodium sulfite and the like, oil-soluble antioxidants such as ascorbyl palmitate, butylated hydroxyanisole (BHA), butylated hydroxytoluene (BHT), lecithin, propyl gallate, alpha-tocopherol, and the like, and metal chelating agents, such as citric acid, ethylenediamine tetraacetic acid (EDTA), sorbitol, tartaric acid, phosphoric acid, and the like.

For topical administration, the preparations may be in the form of a lotion, gel, emulsion, ointment, bioadhesive composition, and the like. Besides EPO, or a functional fragment, derivative or analogue thereof, these preparations may, for instance, comprise one or more of aloe vera, tocopherol acetate, glycerine, stearic acid, 1-hexadecanol, polysorbate 60, apricot kernal oil, glyceryl sterate, PEG-100 stearate, dimethicone, PCP, allantoin, triethanolamine, carboner-940, etc., the general additions known to the skilled person. Further, they may comprise hyaluronic acid and/or a cromolyn compound (U.S. Pat. No. 6,573,249), which are beneficial for treating decubitus ulcers. They may also include anti-inflammatory agents, analgesics, and the like.

The molecules are typically formulated in the compositions and pharmaceutical compositions of the invention in a therapeutically or diagnostically effective amount. Dosage regimens can be adjusted to provide the optimum desired response (e.g., a therapeutic response). Furthermore, for example, a single bolus may be administered, several divided doses may be administered over time, or the dose may be proportionally reduced or increased as indicated by the exigencies of the therapeutic situation. The molecules and compositions, according to the present invention, are preferably sterile. Methods to render these molecules and compositions sterile are well known in the art. The exact dosing regimen is usually sorted out during clinical trials in human patients.

Next to that, kits comprising at least EPO, or a functional fragment, derivative or analogue thereof, for the treatment of decubitus are also a part of the present invention. Optionally, the components of the kits of the invention are packed in suitable containers and labeled for prophylaxis and/or treatment of the indicated conditions. The above-mentioned components may be stored in unit or multi-dose containers, for example, sealed ampules, vials, bottles, syringes, and test tubes, as an aqueous, preferably sterile, solution or as a lyophilized, preferably sterile, formulation for reconstitution. The containers may be formed from a variety of materials such as glass or plastic and may have a sterile access port (for example, the container may be an intravenous solution bag or a vial having a stopper pierceable by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, and syringes. Associated with the kits can be instructions customarily included in commercial packages of therapeutic or prophylactic products that contain information about, for example, the indications, usage, dosage, manufacture, administration, contraindications and/or warnings concerning the use of such therapeutic or prophylactic products.

For the present invention, certain embodiments of a pharmaceutical composition comprise agents used in formulations for topical administration, as described above.

The invention will now be illustrated with the following examples, which are not to be construed to limit the scope of the invention.

### EXAMPLES

In the following examples, the administration of two forms of EPO to the subjects (n=10 per group) is compared to each other, and to a negative control. The negative control is buffer (placebo), the two forms of EPO are commercially available EPO, such as EPREX, and EPO that has been produced on PER.C6 cells (WO 00/63403, WO 03/038100). The dosage used is 100 to 5000 IU/kg. The EPO is administered intravenously or locally, i.e., subcutaneously at the site of the pressure. EPO is administered just before, during or preferably directly after the release of every episode of the pressure.

### EXAMPLE 1

Model of Chronic Pressure Ulcer in the Rat

In this model, chronic pressure ulcers are induced by a device consisting of a subcutaneously implanted ferromagnetic steel plate and an externally applied magnet as described in S. M. Pierce et al. (2000), Wound Rep. Reg. 8:68-76.

EPO or placebo is administered systemically or locally after completion of each pressure application. The damage in the skin and subcutaneous tissues is quantitatively estimated at fixed time points after the termination of the pressure applications. The parameters recorded are blood flow in the pressurized area, the size of the necrotic area that develops and histological determinants of inflammation and necrosis in the skin and underlying tissues. It is expected that the injury measured in the groups receiving EPO will be less than in the groups receiving placebo treatment.

### EXAMPLE 2

A Different Model of Chronic Pressure Ulcer in Hairless Rats

In this model, pressure is applied on the skin area over the trochanter major of the rat. The surface pressure is computer controlled according to the methods described in R. Salcido et al. (1995), J. Rehab. Res. Develop. 32:149-161). The administrations of EPO and placebo, as well as the measured endpoints, are similar to those described in Example 1. It is expected that in the animals treated with EPO, the degree of injury will be reduced as compared to the animals given placebo.

### EXAMPLE 3

Pressure-Induced Skin Injury in Pigs

In this model, pressure is applied on discrete areas of the dorsal skin of pigs over the femoral trochanters and damage is measured histologically at various times after the start of reperfusion according to methods described in R. Houwing et al. (2000), J. Wound Care 9:36-44). In this case, the test compounds will be administered systemically or locally before or after the completion of pressure application. It is expected that tissue injury will be less following treatment with EPO as compared to treatment with placebo.

### EXAMPLE 4

New Model for Decubitus: Pressure-Induced Ulcers in Mice

In this model, pressure is applied to the dorsal skin and subcutaneous tissues of mice by positioning two magnetic discs, one on each opposing side of a skin fold for a certain time period. The magnetic discs are held in place by the magnetic force which is equivalent to a pressure on the skin fold of approximately 450 kPa. The surface of the discs is circular with a diameter of 8 mm. The mice can be of the hairless type, such as mice of the strain MF-1 and KH, or mice with a normal pelt. In the latter case, the skin is shaved with an electrical pair of clippers prior to the application of the magnetic discs.

The magnetic pressure device is held in place for a predetermined period of time by the magnetic force on the unanesthetized animals.

After the magnets have been removed, the tissue that was pressurized between the magnets appears pale and bloodless. By intravenous injection of a vital dye into these mice, it was demonstrated that the circulation in the previously compressed tissue had been disrupted. It also appears from the same dye technique, that reperfusion of these tissues is slowly re-established within about 30 minutes. These observations demonstrate that the induction of pressure ulcers by this method is due to ischemia/reperfusion, similar to the mechanism assumed to prevail in the causation of clinical bed sores.

The next few days, the skin in the compressed areas on the back of the mouse, one at each side of the dorsal midline, is yellowish and erythematous and develops crusts. By day 3 or 4 after release of the pressure, these areas become dark brown and take the appearance of ulcers.

Histological examination of these wounds confirmed the presence of epidermolysis, necrosis and inflammation of the underlying tissues, which penetrates into the muscle layer of the panniculus. In all aspects, these wounds resemble those of severe pressure ulcers, but bone is obviously absent from the deeper layers of the lesion.

Between day 4 and 7, the surface of the ulcers may become moist from wound exudate, and crust formation continues. The surface of the ulcers was measured with vernier callipers and daily measurements revealed that the ulcer area begins to decrease at day 4 to 5. Depending on the duration of exposure to pressure, the ulcers have completely healed between 10 and 16 days after release of pressure in the sense that complete re-epithelialization of the skin has occurred, and that the deeper layers have been replaced by scar tissue.

### EXAMPLE 5

Treatment with EPO Decreases the Size of Pressure Ulcers

Using the quantification of the surface area of the lesions as described in Example 4, it was shown in three different experiments that treatment with EPO decreases the size of the resulting ulcers.

The EPO that was used in the following experiments was produced in PER.C6 cells (as deposited at the ECACC under no. 96022940), and has a specific glycosylation pattern (WO 03/038100). In particular, the EPO used for these experiments is characterized by the presence of Lewis-X (and/or sialyl-Lewis-X, which are included in the definition of Lewis-X) structures on the N-linked glycans and, in particular, the presently used EPO contained around 0.6 Lewis-X structure per N-linked glycan (EPO contains 3 N-linked glycans). It contains about five sialic acids per molecule (for comparison: the commercially available EPREX contains about 12.1 sialic acids per molecule, when measured using the same analysis method (based on quantification of an iso-electric focusing gel)). For comparison, the EPO as produced in PER.C6 cells and used for these experiments appears at least about 25 times less active in red blood cell production than commercially available EPREX.

A. In the first experiment, hairless MF-1 female mice were exposed to the magnetic pressure device described in Example 4 for eight hours. A group of four mice was treated twice with 3.3 μg of EPO. Treatment was by intraperitoneal injection of EPO in a volume of 0.5 ml at one hour before the application of pressure and immediately after the release of the pressure. A control group of six mice was given similar treatment with the solvent only.

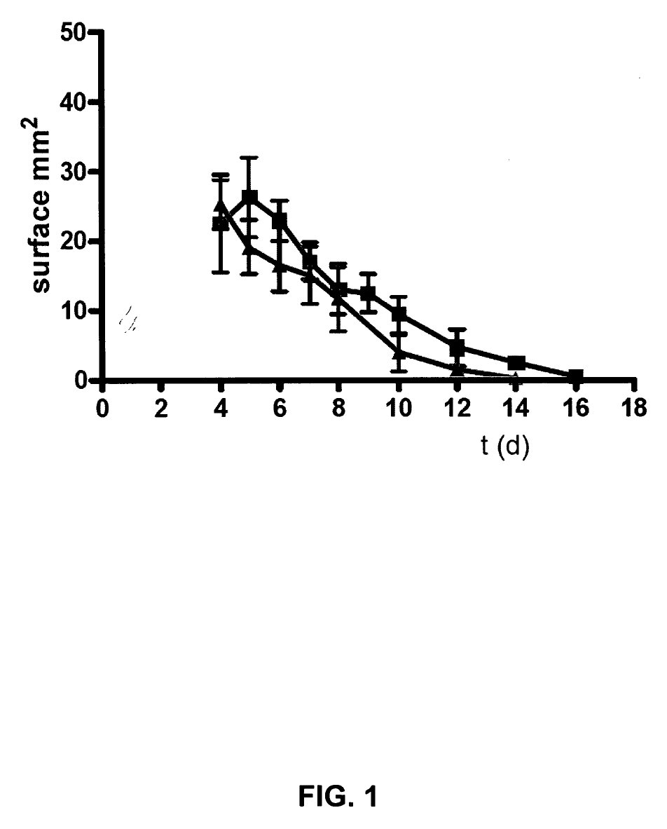

The results are depicted in FIG. 1. It shows that the curve for the EPO-treated mice runs below that of the controls, suggesting smaller lesions upon EPO treatment, but the difference is not statistically significant (p=0.18) when the data are subjected to analysis of repeated measures.

B. The second experiment was performed with Balb/c female mice, which have a normal pelt. The shaved dorsal skin fold was subjected to pressure for ten hours. One group of eight mice was treated with 4.5 μg of EPO. Treatment was by intraperitoneal injection of EPO in a volume of 0.5 ml at one hour before the application of pressure. A control group of eight mice was given similar treatment with the solvent only.

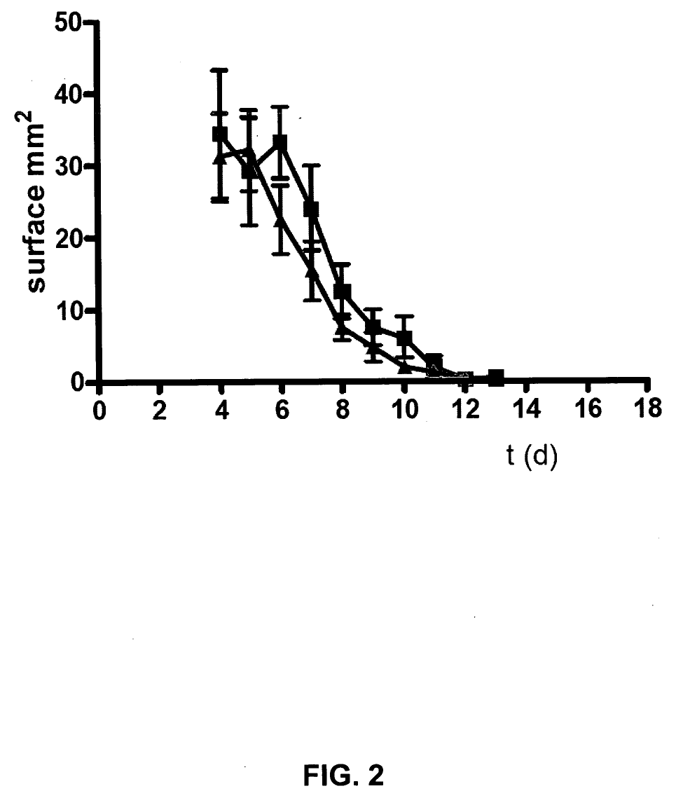

The results are depicted in FIG. 2. Again, the curve for the EPO-treated mice runs below that of the controls. Using analysis of repeated measures, the difference in size between the EPO-treated and the control mice is statistically significant (p=0.04).

C. In a third experiment, hairless MF-1 mice were used and the treatment was performed by intravenous injection of EPO given ten minutes before the application of the magnetic device. The pressure was maintained for a period of six hours. One group of three females and three males was injected with 4.5 μg of EPO using an injection volume of 0.5 ml. The control group consisting of three females and four males received similar treatment with the solvent only.

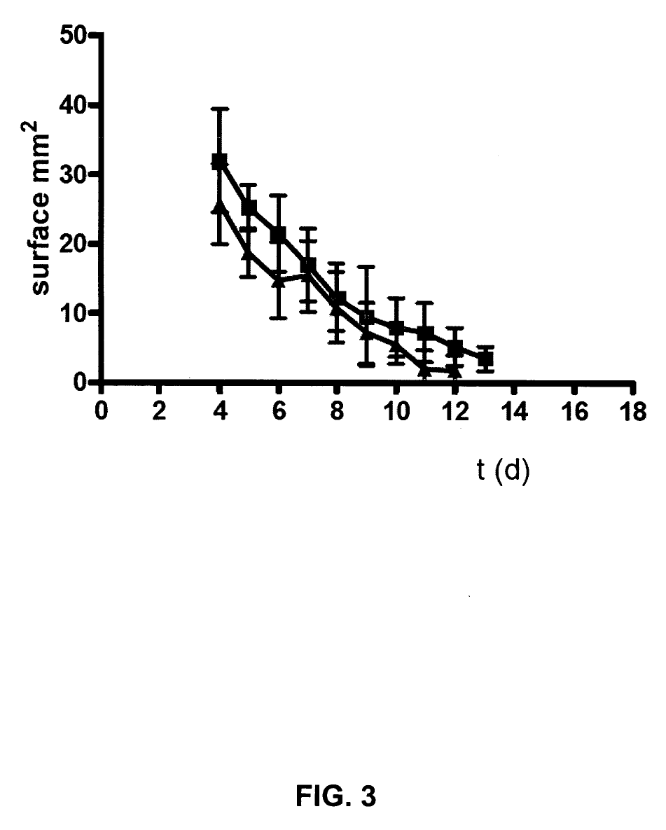

The results are depicted in FIG. 3. As in the previous experiments, the average surface area of the ulcers of the EPO-treated mice is smaller than that of the controls. The difference closely approaches statistical significance (p=0.06) according to analysis of repeated measures.

When all data of the three experiments described above are subjected together to an analysis of repeated measures, the ulcers of the EPO-treated mice are on the average 3.3 mm² smaller and this difference is highly significant (p=0.01).

These findings indicate that EPO treatment provides protection against pressure ulcer formation in that the resulting ulcers are smaller than without treatment. The graphs also show that, as a consequence of the smaller ulcer size, the time required for healing of the ulcers, i.e., to achieve complete re-epithelialization is reduced by two to three days.

What is claimed is:

1. A method of treating a subject suffering from, or at risk of suffering from, decubitus, the method comprising:
administering erythropoietin, or a functional part, derivative or analogue thereof to the subject.

2. The method according to claim 1, wherein the subject is not anemic.

3. The method according to claim 1, wherein the subject is a human subject.

4. The method according to claim 2, wherein the subject is a human subject.

5. The method according to claim 1, wherein administration occurs during stage I of clinical symptoms associated with decubitis.

6. The method according to claim 2, wherein administration occurs during stage I of clinical symptoms associated with decubitis.

7. The method according to claim 3, wherein administration occurs during stage I of clinical symptoms associated with decubitis.

8. The method according to claim 4, wherein administration occurs during stage I of clinical symptoms associated with decubitis.

9. The method according to claim 1, wherein administration occurs before clinical symptoms of decubitis are observed.

10. The method according to claim 2, wherein administration occurs before clinical symptoms of decubitis are observed.

11. The method according to claim 3, wherein administration occurs before clinical symptoms of decubitis are observed.

12. The method according to claim 4, wherein administration occurs before clinical symptoms of decubitis are observed.

13. A method of preventing decubitis in a subject in need thereof, the method comprising:
administering to the subject erythropoietin or a functional part, derivative or analogue thereof so as to prevent decubitus.

15. A pharmaceutical preparation for the treatment of decubitus ulcers, characterized in that said preparation comprises:
a) erythropoietin, or a functional part, derivative or analogue thereof; and
b) an analgesic and/or an anti-inflammatory agent.

16. The pharmaceutical preparation of claim 15, wherein said erythropoietin has been recombinantly produced in host cells that further express the E1A protein of an adenovirus.

17. The pharmaceutical preparation of claim 16, wherein said host cells are derived from primary human retina cells.

18. The pharmaceutical preparation of claim 17, wherein said host cells are derived from PER.C6 cells.

19. The pharmaceutical preparation of claim 16, wherein said erythropoietin comprises, on average, at least 1.5 Lewis-X structure per erythropoietin molecule.

20. The pharmaceutical preparation according to claim 16, wherein said erythropoietin comprises on average less than ten sialic acid moieties per erythropoietin molecule.
